(19) United States
(12) Patent Application Publication
(10) Pub. No.: US 2021/0393669 A1
(43) Pub. Date: Dec. 23, 2021

(54) METHODS AND COMPOSITIONS FOR TREATING HEMOPHILIA

(71) Applicant: Genzyme Corporation, Cambridge, MA (US)

(72) Inventors: Baisong Mei, Waban, MA (US); Shauna Andersson, Woburn, MA (US); Qifeng Yu, Shrewsbury, MA (US); Pronabesh DasMahapatra, Somerville, MA (US)

(73) Assignee: Genzyme Corporation, Cambridge, MA (US)

(21) Appl. No.: 17/354,818

(22) Filed: Jun. 22, 2021

Related U.S. Application Data
(60) Provisional application No. 63/042,390, filed on Jun. 22, 2020.

(51) Int. Cl. A61K 31/713; A61P 7/04; A61P 19/02

(52) U.S. Cl. CPC A61K 31/713; A61P 19/02; A61P 7/04

(57) ABSTRACT

The present disclosure provides using a double-stranded oligonucleotide compound as a novel therapy to improve the quality of life and joint function of patients with hemophilia A and hemophilia B.

### CROSS-REFERENCE TO RELATED APPLICATION

This application claims priority from U.S. Patent Application No. 63/042,390, filed Jun. 22, 2020, the disclosure of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Sep. 3, 2021, is named 022548.US080_SL_rev1.txt and is 772 bytes in size.

### BACKGROUND OF THE INVENTION

Maintenance of normal hemostasis relies on a regulated set of simultaneously occurring procoagulant and anticoagulant processes, in which thrombin plays a central role. Hemophilia A and B are inherited bleeding disorders characterized by the body's inability to control blood clotting. They are caused by deficiencies in factors VIII and IX, respectively. Bleeding in hemophilia A and B arises from insufficient thrombin generation (Peyvandi et al., Lancet (2016) 388(10040):187-97). Without effective treatment, patients with hemophilia experience recurrent bleeding, which lead to major disability due to chronic hemarthropathy and significant pain, and can be life-threatening (Pipe et al., Haemophilia (2007) 13 Suppl 4:1-16).

Significant unmet needs and management challenges continue to exist for all hemophilia populations despite treatment advances. While prophylaxis based on replacement therapy with factor VIII or IX is considered the cornerstone of hemophilia management, it has significant limitations. For example, injections of factor replacement for prophylaxis are burdensome and impractical, often requiring multiple intravenous infusions per week (Peyvandi, supra; Ljung and Andersson, Br J Haematol. (2015) 169(6):777-86; Srivastava et al., Haemophilia (2013) 19(1):e1-47; Bauer, Am J Manag Care (2015) 21(6 Suppl):S112-22; Mannucci and Franchini, Blood Transfus. (2013) 11(Suppl 4):s77-81). Factor replacement is also limited by difficulties with venous access and risk of infections (Balkaransingh and Young, Ther Adv Hematol. (2018) 9(2):49-61; Valentino et al., Blood Rev. (2011) 25(1):11-5). Limitations in delivering factor replacement also result in a large proportion of the world's hemophilia population without access in the first instance to prophylaxis treatment (Hemophilia, W.F.O. Treatment Safety and Supply. 2020).

Additionally, treatment with factor replacement products can result in the development of inhibitory alloantibodies, rendering the factor treatment ineffective (Morfini et al., Haemophilia (2007) 13(5):606-12). These inhibitors, which typically occur in childhood, limit treatment options and dramatically worsen the prognosis of hemophilia. Moreover, those with persistent inhibitors typically have a lower quality of life, greater joint disease, greater surgical risk, and higher mortality, including a higher risk of death from hemophilia-related bleeding complications, when compared to patients without inhibitors (Morfini, supra; Oladapo et al., Orphanet J Rare Dis. (2018) 13(1):198). Current treatment strategies for individuals with persistent inhibitors include immune tolerance induction (ITI) and prophylaxis with bypassing agents (BPAs) such as activated prothrombin complex concentrates (aPCC) and recombinant activated factor VII (rFVIIa) (Benson et al., Eur J Haematol. (2012) 88(5):371-79; Collins et al., Br J Haematol. (2013) 160(2):153-70; Kempton et al., Blood (2014) 124(23):3365-72; Astermark et al., Haemophilia (2007) 13(1):38-45; Eichinger et al., Eur J Clin Invest. (2009) 39(8):707-13).

There remains an urgent and unmet need to develop therapeutics and treatment methods that prevent recurrent bleeding and improve the overall quality of life for patients with hemophilia.

### SUMMARY OF THE INVENTION

The present disclosure provides methods and compositions for treating hemophilia patients. In one aspect, the disclosure provides a method of improving joint function in a hemophilia patient (e.g., a hemophilia A or B patient with or without inhibitors) in need thereof, comprising administering (e.g., subcutaneously) fitusiran at 40-90 mg per dose to the patient. In some embodiments, the treatment reduces difficulty in walking or increases mobility.

In one aspect, the present disclosure provides a method of improving a joint symptom (e.g., joint swelling, painful movement, and joint pain) in a hemophilia patient (e.g., a hemophilia A or B patient with or without inhibitors) in need thereof, comprising administering (e.g., subcutaneously) fitusiran at 40-90 mg per dose to the patient.

In one aspect, the present disclosure provides a method of improving patient-reported outcome (PRO) in a hemophilia patient (e.g., a hemophilia A or B patient with or without inhibitors) in need thereof, comprising administering (e.g., subcutaneously) fitusiran at 40-90 mg per dose to the patient. In some embodiments, the PRO is improved in one or more quality-of-life (QoL) domains.

In one aspect, the present disclosure provides a method of improving QoL in a hemophilia patient (e.g., a hemophilia A or B patient with or without inhibitors) in need thereof, comprising administering (e.g., subcutaneously) fitusiran at 40-90 mg per dose to the patient in need thereof, wherein the QoL is improved in one or more QoL domains.

In some embodiments, the one or more QoL domains are domains in a QoL questionnaire (e.g., Haemophilia Quality of Life Questionnaire for Adults (Haem-A-QoL)). In further embodiments, the treated patient experiences a clinically meaningful improvement indicated by a reduction of 7 or more units (e.g., 8 or more, 9 or more, or 10 or more units) in one or more of scores (e.g., Total Score, Sports and Leisure domain score, and Physical Health domain score) of the Questionnaire.

In some embodiments, the patient is an adult or adolescent patient twelve years or older with hemophilia A or B (congenital factor VIII or factor IX deficiency) with or without inhibitors.

In some embodiments, the patient has hemophilia A. In further embodiments, the patient has been treated with factor VIII replacement or a bypassing agent (BPA; e.g., aPCC or rFVIIa). In certain embodiments, the patient is with inhibitors (e.g., with a level of inhibitors more than 0.6 BU/mL as determined by Bethesda inhibitor assay). In other embodiments, the patient is without inhibitors.

In some embodiments, the patient has hemophilia B. In further embodiments, the patient has been treated with factor IX replacement or a BPA (e.g., aPCC or rFVIIa). In certain embodiments, the patient is with inhibitors (e.g., with a level of inhibitors more than 0.6 BU/mL as determined by Bethesda inhibitor assay). In other embodiments, the patient is without inhibitors.

In some embodiments, the patient is treated with a plurality of doses of fitusiran at 50 mg per dose, or with a plurality of doses of fitusiran at 80 mg per dose. In some embodiments, fitusiran is administered to the patient once every four weeks or once a month. In some embodiments, fitusiran is provided in a phosphate-buffered saline (pH 7) at 50-200 mg/mL, optionally 100 mg/mL.

Also provided in the present disclosure is an article of manufacture for use in the present treatment methods. In some embodiments, the article of manufacture is a single-use vial containing 80 mg of fitusiran in 0.8 mL of a phosphate-buffered saline (pH 7). In other embodiments, the article of manufacture is a single-use prefilled syringe containing 80 mg of fitusiran in 0.8 mL of a phosphate-buffered saline (pH 7).

The present disclosure also provides the use of fitusiran for the manufacture of a medicament to treat hemophilia in the present treatment methods, as well as fitusiran for use in the present treatment methods.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIG. 1 shows the expanded structural formula, chemical formula, and molecular mass of fitusiran.

FIG. 2 is a CONSORT diagram showing the design of the fitusiran clinical study described herein.

FIGS. 3A and 3B show the D-dimer (μg/mL) levels over time per participant by dose group. A: 50 mg dose group. B: 80 mg dose group.

FIG. 4A is a graph showing mean (±standard error of the mean [SEM]) antithrombin (AT) activity of fitusiran relative to baseline in patients with hemophilia A or B with inhibitors receiving fitusiran monthly. MDI: multiple dose with inhibitors.

FIG. 4B is a graph showing mean (±SEM) of plasma thrombin generation peak height (nmol/L) over time in patients of the 50 mg and 80 mg dose groups.

FIG. 5 shows post hoc analysis of thrombin generation associated with AT reduction in patients with hemophilia A or B with inhibitors. For all patients, at each time point an AT level and a corresponding thrombin generation measurement were recorded. All available thrombin generation values were associated with an AT activity level relative to baseline and were binned into AT lowering quartiles. Middle line in the boxes denote median values and top and bottom of the boxes represent the interquartile ranges. Minimum and maximum values are shown by the bars (excluding outliers). Healthy volunteer data (Pasi et al., N Engl J Med. (2017) 377(9):819-28) were used as a reference.

### DETAILED DESCRIPTION OF THE INVENTION

The present disclosure features methods of using fitusiran for routine prophylaxis to prevent or reduce the frequency of bleeding episodes in adult and adolescent patients (≥12 years old) with hemophilia, such as hemophilia A (congenital factor VIII deficiency) or hemophilia B (congenital factor IX deficiency), with or without inhibitors. These methods reduce joint swelling and/or joint pain, improve joint function, and improve the quality of life of the patients. In particular embodiments, the present methods improve the patients' quality-of-life scores such as those associated with physical health.

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods are beneficial for hemophilia A patients with or without inhibitors, as well as for hemophilia B patients with or without inhibitors.

### I. Fitusiran Pharmaceutical Compositions

The structure of fitusiran is provided herein. Fitusiran is a synthetically, chemically modified double-stranded small interfering RNA (siRNA) oligonucleotide covalently linked to a tri-antennary N-acetyl-galactosamine (GalNAc) ligand targeting the AT3 mRNA in the liver, thereby suppressing the synthesis of antithrombin. See, e.g., Pasi, supra. Antithrombin is encoded by the SERPINC1 gene. The nucleosides in each strand of fitusiran are connected through 3′-5′ phosphodiester linkages, thus forming the sugar-phosphate backbone of the oligonucleotide.

The sense strand and the antisense strand contain 21 and 23 nucleotides, respectively. The 3′-end of the sense strand is conjugated to the GalNAc containing moiety (referred to as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. No. 9,127,274, US20170159053, and WO 2019/014187.

The two nucleotide strands of fitusiran are shown below:
sense strand: 5′ Gf-ps-Gm-ps-Uf-Um-Af-Am-Cf-Am-Cf-Cf-Af-Um-Uf-Um-Af-Cm-Uf-Um-Cf-Am-Af-L96 3′ (SEQ ID NO: 1), and
antisense strand: 5′ Um-ps-Uf-ps-Gm-Af-Am-Gf-Um-Af-Am-Af-Um-Gm-Gm-Uf-Gm-Uf-Um-Af-Am—Cf-Cm-ps-Am-ps-Gm 3′ (SEQ ID NO:2),
wherein
Af=2′-fluoroadenosine
Cf=2′-fluorocytidine
Gf=2′-fluoroguanosine
Uf=2′-fluorouridine
Am=2′-O-methyladenosine
Cm=2′-O-methylcytidine
Gm=2′-O-methylguanosine
Um=2′-O-methyluridine
“-” (hyphen)=3′-5′ phosphodiester linkage sodium salt
“-ps-”=3′-5′ phosphorothioate linkage sodium salt
and wherein L96 has the following formula:

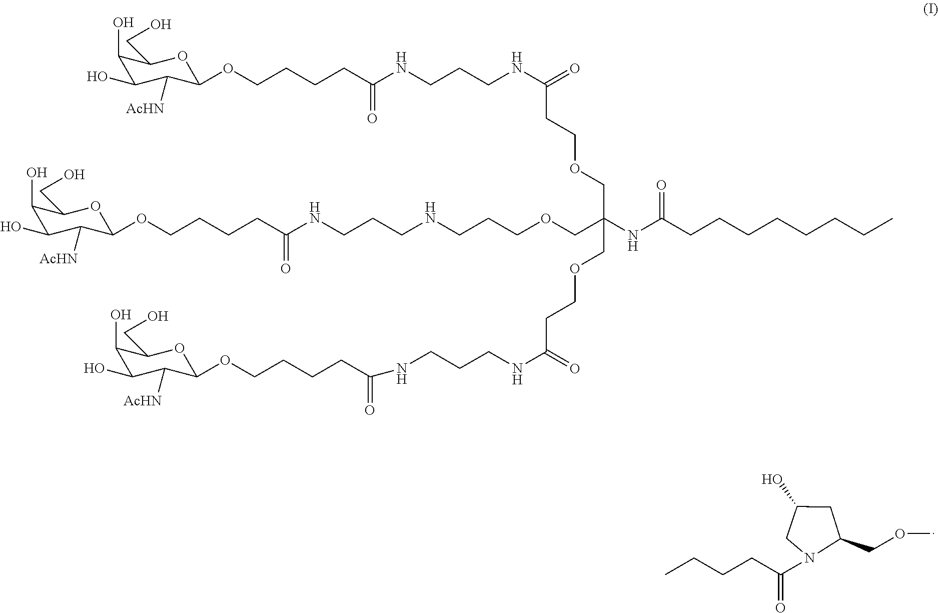

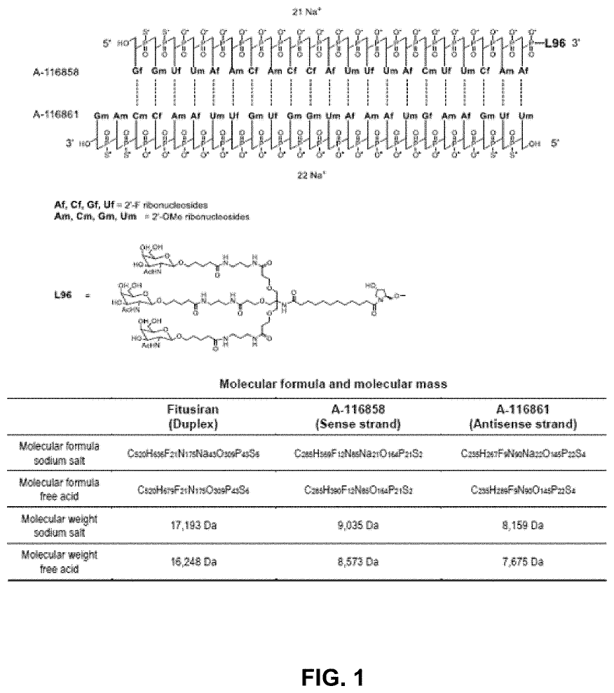

The expanded structural formula, molecular formula, and molecular weight of fitusiran are shown in FIG. 1.

For use in the present treatment methods, fitusiran may be provided in a pharmaceutical composition comprising it and a pharmaceutically acceptable excipient. In certain embodiments, the dsRNA compound is in sodium salt form.

In some embodiments, fitusiran is provided in an aqueous solution at a concentration of 50 to 200 mg/mL (e.g., 50 to 150 mg/mL, 80 to 110 mg/mL, or 90 to 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical composition comprises fitusiran at a concentration of 50, 75, 100, 125, 150, or 200 mg/mL.

Unless otherwise indicated, a fitusiran weight recited in the present disclosure is the weight of fitusiran free acid (the active moiety). For example, 100 mg/mL fitusiran means 100 mg of fitusiran free acid (equivalent to 106 mg fitusiran sodium, the active substance) per mL.

In some embodiments, the pharmaceutical compositions comprise fitusiran in a phophate-buffered saline. The phosphate concentration in the solution may be 1 to 10 mM (e.g., 2, 3, 4, 5, 6, 7, 8, or 9 mM), with a pH of 6.0-8.0. The pharmaceutical compositions herein may include a preservative such as EDTA. Alternatively, the pharmaceutical compositions are preservative-free. In particular embodiments, the fitusiran pharmaceutical composition is preservative-free and comprises, consists of, or consists essentially of 100 mg of fitusiran per mL of a 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the composition to 7.0.

The pharmaceutical composition may be provided in a single-use container (e.g., a vial, an ampule, a syringe, or an injector), with each container containing 40-100 mg fitusiran (e.g., 50 mg or 80 mg). The fitusiran may be provided in a solid form in the container and reconstituted in an aqueous solution (e.g., PBS) prior to use, with the reconstituted solution containing 50-150 mg/mL (e.g., 100 mg/mL) fitusiran. In some embodiments, fitusiran is provided in sodium form in a single-use glass vial or a single-use prefilled syringe (e.g., one with a safety system). In further embodiments, each vial or syringe contains 80 mg of fitusiran in 0.8 mL of 5 mM phosphate buffered saline solution (pH 7.0); and the solution is administered to patients through subcutaneous injection. The solution can be stored at 2 to 30° C. (e.g., 2 to 8° C.).

In particular embodiments, the fitusiran composition for subcutaneous injection contains fitusiran in a 5 mM phosphate buffered saline having 0.64 mM NaH₂PO₄, 4.36 mM Na₂HPO₄, and 84 mM NaCl at pH 7.0. In certain embodiments, the composition of fitusiran solution for subcutaneous injection is shown in Table 1 below:

TABLE 1

| | Composition | | | |
|---|---|---|---|---|
| | | | Per unit | Per unit |
| | Percentage | Per ml | (2 mL vial) | (1 mL syringe) |
| Components | [%] | [mg] | [mg] | [mg] |
| Fitusiran (active moiety) | 10 | 100 | 80 | 80 |
| [equivalent to fitusiran | | [106] | [84.8] | [84.8] |
| sodium] | | | | |
| Sodium chloride | 0.49 | 4.909 | 3.927 | 3.927 |
| Dibasic sodium phosphate | 0.12 | 1.169 | 0.935 | 0.935 |
| (heptahydrate) | | | | |
| Monobasic sodium phosphate | <0.01 | 0.0885 | 0.0708 | 0.0708 |
| (monohydrate) | | | | |
| Phosphoric acid, concentrated | — | q.s. pH 7.0 | q.s. pH 7.0 | q.s. pH 7.0 |
| Sodium hydroxide | — | q.s. pH 7.0 | q.s. pH 7.0 | q.s. pH 7.0 |
| Water for injection | q.s. 100 | q.s. 1 mL | q.s. 0.8 mL | q.s. 0.8 mL |

q.s.: quantum satis.

### II. Therapeutic Use of Fitusiran

Fitusiran can suppress liver production of antithrombin (AT). In its role as an anti-coagulant, AT regulates hemostasis by directly targeting thrombin production or by inactivating uncomplexed FXa, which in turn reduces thrombin production (Quinsey et al., Int J Biochem Cell Biol. (2004) 36(3):386-9). Fitusiran may be used to treat those who have impaired hemostasis.

For example, fitusiran can be used to treat patients with hemophilia A or B with or without inhibitors for routine prophylaxis to prevent or reduce the frequency of bleeding episodes. In particular embodiments, fitusiran is used to treat adult and adolescent patients (≥12 years old) with hemophilia A or B (congenital factor VIII or factor IX deficiency) with or without inhibitors.

The present methods include administering to the hemophilia patient (e.g., a hemophilia A or hemophilia B patient) in need thereof a therapeutically effective amount of fitusiran. “Therapeutically effective amount” refers to the amount of fitusiran that helps the patient to achieve a desired clinical endpoint. A desired clinical endpoint may be, for example, reduction of annual bleeding rates (ABR) (e.g., by more than 10, 20, 30, 40, 50, 60, 70, 80, 90, or 100%). A desired clinical endpoint may be reduction of antithrombin levels in the patient to a normal level (e.g., about 64-210 nM).

A desired clinical endpoint may also be improved patient-reported outcomes (PROs) as further described below. In some embodiments, the treatment efficacy can be measured by a reduction in the severity of disease as evaluated by the patient based on a valid and reliable hemophilia-specific PRO instrument, for example, the Haemophilia Quality of Life Questionnaire for Adults (“Haem-A-QoL”; von Mackensen et al., Haematologica (2005) 90(52):115-6, Abstract 0290; Wyrwich et al., Haemophilia (2015) 21(5):578-84). Any positive change resulting in, for example, lessening of severity of disease measured using the appropriate scale, represents adequate treatment using the pharmaceutical compositions as described herein.

Hemophilia, through its associated symptoms, functional limitations and treatment burden, directly impacts the health-related quality of life (HRQoL) of patients. Improving HRQoL is a critical aspect of hemophilia disease management. The present methods improve HRQoL of patients as determined by well-designed, detailed questionnaires. For examples, HRQoL in adult patients (17 years or older, e.g., 18 years or older) can be measured by questionnaires Hemofilia-QoL, Haemophila Well-Being Index, HAEMO-QoL-A, Haem-A-QoL, and EuroQol 5-Dimensions (EQ-5D) (EuroQol Group, Health Policy (1990) 16(3):199-208). HRQoL in adolescent patients (12 years or older to 17 years old) can be measured by, e.g., Haemophilia Quality of Life Questionnaire for teenagers (Haemo-QoL). See, e.g., Bullinger et al., Value Health. (2009) 12(5):808-20; and Remor, Int J Behav Med. (2013) 20(4):609-17.

In some embodiments, the present treatment improves the quality of life of patients in one or more of QoL domains (e.g., hemophilia-specific QoL domains). These QoL domains include, for example, domains related to Physical Health, Feeling, View of Self, Sports and Leisure, Work and School, Dealing with Hemophilia, Treatment, Future, Family Planning, and/or Partnership and Sexuality. Improvement in these domains may be evaluated by patient-reported outcome (PRO) and may be aided by questionnaires. For example, improvement in these domains may be reported by a patient to his/her physician, and/or may be scored by a QoL questionnaire.

In particular embodiments, HRQoL of adult patients is measured by scores in Haem-A-QoL. See, e.g., von Mackensen et al., Value in Health. (2005) 8(6):A127; von Mackensen et al., J Thrombosis and Haemostasis. (2005) 3(Sup1):P0813; von Mackensen and Gringeri, “Quality of Life in Hemophilia” In: Handbook of Disease Burdens and Quality of Life Measures. Heidelberg: Springer; 2009, pp. 1910-1; and Bullinger et al., Value in Health. (2009) 12(5):808-20; Wyrwich, supra. The Haem-A-QoL questionnaire includes 46 items contributing to 10 domains, including Physical Health (5 items), Feeling (4 items), View of Self (5 items), Sports and Leisure (5 items), Work and School (4 items), Dealing with Hemophilia (3 items), Treatment (8 items), Future (5 items), Family Planning (4 items), and Partnership and Sexuality (3 items).

All Haem-A-QoL items are measured based on a 5-point frequency scale (1=never, 2=rarely, 3=sometimes, 4=often, and 5=all the time). A “Not Applicable” response option is also available for the domains of “Sports & Leisure,” “Work & School,” and “Family Planning” when the question does not apply to the participant. A “Total Score” is also used to represent the average of all 10 domains of the Haem-A-QoL questionnaire. Haem-A-QoL domain scores and the Total Score are transformed to a scale of 0-100 with higher scores representing greater impairment. A decrease in score relative to the corresponding baseline score (score before the treatment being evaluated) indicates an improvement in the patient's quality of life. The questionnaire may be taken before treatment and after treatment with one or more (e.g., two or more, three or more, four or more, five or more, or six or more) doses of fitusiran (e.g., administered subcutaneously at 80 mg once every four weeks or once a month). For example, the questionnaire may be taken at week 8, 12, 16, 20, 24, 25, 26, or 27 after commencement of fitusiran treatment.

The present fitusiran therapy improves the score from at least one of the Haem-A-QoL domains (e.g., Physical Health, Feeling, View of Self, Sports and Leisure, Work and School, Dealing with Hemophilia, Treatment, Future, Family Planning, and/or Partnership and Sexuality) from baseline, and/or improves the Haem-A-QoL Total Score from baseline. In particular, the present methods may improve the quality of life of hemophilia patients, including improvement (e.g., alleviation and disappearance) of patient-reported hemophilia-related symptoms (e.g., painful swellings and joint pain) and physical functioning (e.g., pain with movement and difficulty walking far) as determined by the Physical Health score and/or the Total Score of Haem-A-QoL. A clinically meaningful improvement of quality of life includes, for example, an about 7 or more point reduction in the Total Score, an about 10 or more point reduction in the Sports and Leisure domain score, and/or an about 10 or more point reduction in the Physical Health domain score. See Wyrwich, supra. In some embodiments, one or more of the 10 domain scores (e.g., the Physical Health domain score or the Total Score) in Haem-A-QoL is reduced by 1 or more units (e.g., 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more units).

### III. Administration of Fitusiran Pharmaceutical Compositions

The fitusiran pharmaceutical composition may be administered by any means known in the art including, but not limited to, intraperitoneal, intravenous, intramuscular, subcutaneous, transdermal, or hepatic portal vein administration. In certain embodiments, the pharmaceutical composition is administered by subcutaneous injection at a dose strength of, for example, 25 to 100 mg (e.g., 25 to 95 mg, 40 to 90 mg, 50 to 100 mg, 50 to 90 mg, 50 to 85 mg, or 50 to 80 mg) per dose. In particular embodiments, fitusiran is administered subcutaneously at 50 or 80 mg (weight of active moiety) per dose in a PBS solution as described above.

A plurality of fitusiran doses may be administered to a subject at an interval of 1, 2, 3, 4, 5, 6, 7, or 8 weeks, or of 1, 2, or 3 months. In particular embodiments, a fixed dose of fitusiran (e.g., 50 or 80 mg subcutaneous injection) is administered to a hemophilia patient (e.g., a hemophilia A or hemophilia B patient who is twelve years or older and who has or has not developed inhibitors) once every four weeks or once a month.

In some embodiments, the present pharmaceutical compositions can be administered with other pharmaceuticals and/or other therapeutic methods, such as those that are currently employed for treating bleeding disorders. For example, in certain embodiments, fitusiran is administered in combination with a second agent useful in treating hemophilia A and/or B. Examples of such second agents are fresh-frozen plasma (FFP); rFVIIa; aPCC; recombinant or plasma-derived FVIII or FIX; virus-inactivated, vWF-containing FVIII concentrates; desensitization therapy which may include large doses of FVIII or FIX, along with steroids or intravenous immunoglobulin (IVIG) and cyclophosphamide; plasmapheresis in conjunction with immunosuppression and infusion of FVIII or FIX, with or without antifibrinolytic therapy; immune tolerance induction (ITI), with or without immunosuppressive therapy (e.g., cyclophosphamide, prednisone, and/or anti-CD20); desmopressin acetate (DDAVP); antifibrinolytics, such as aminocaproic acid and tranexamic acid; antihemophilic agents; corticosteroids; immunosuppressive agents; and estrogens. The fitusiran composition and the additional therapeutic agent and/or treatment may be administered at the same time and/or in the same combination, e.g., parenterally, or the additional therapeutic agent can be administered as part of a separate composition or at separate times and/or by another method known in the art or described herein.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of hematology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### EXAMPLES

### Example 1: Clinical Study Design and Population

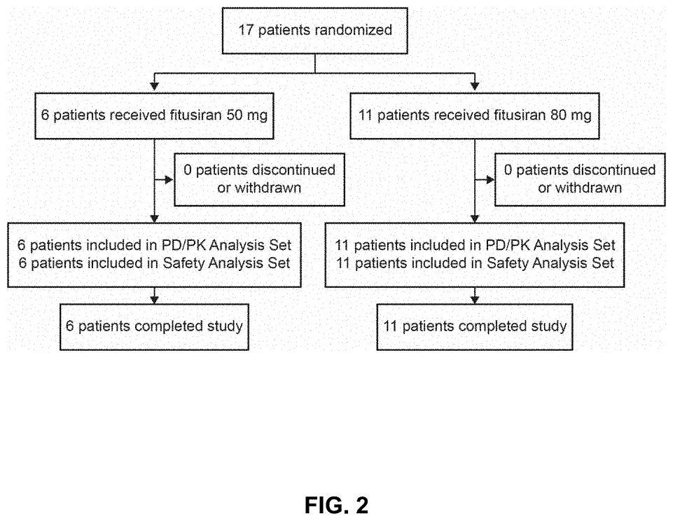

This example describes the design and patient population of a clinical study on fitusiran therapy. In the study, seventeen adults with hemophilia A or B with inhibitors received three monthly fixed subcutaneous doses of fitusiran at 50 mg (n=6) or 80 mg (n=11) (FIG. 2). Participants were followed for up to 112 days (or up to 84 days for those who transitioned to an open-label extension study) or until AT levels returned to ≥80% of the baseline value, whichever period was longer. Bleeding episodes were managed during the study with rFVIIa or aPCC therapy. All participants completed the study.

Eligible subjects were male and aged 18-65 years (inclusive), with moderate or severe hemophilia A or hemophilia B (FVIII or FIX≤5%) with inhibitors (Bethesda inhibitor assay >0.6 BU/mL). Participants had received on-demand treatment or if previously on prophylactic therapy.

Key exclusion criteria included a history of venous thromboembolism, a known coexisting thrombophilic disorder, D-dimer >3.0× upper limit of normal (ULN) at screening, AT activity <60% at screening, liver dysfunction, HIV positive with a CD4 count <200 cells/μL, or an estimated glomerular filtration rate ≤45 mL/min/1.73 m² (using the Modification of Diet in Renal Disease formula) (Levey et al., Ann Intern Med. (2006) 145(4):247-54).

Statistical analyses were primarily descriptive and performed using SAS software, version 9.2 or higher. Descriptive statistics were presented for continuous variables, and frequencies and percentages for categorical and ordinal variables. Percentages were based on the number of non-missing values. The ABR in each period was calculated as the number of bleeds in the period divided by the number of days in that period, multiplied by 365.25.

Baseline demographics and clinical characteristics of the study population are shown in Table 2. SEM refers to standard error of the mean.

TABLE 2

| | 50 mg | | 80 mg | | All treated | |
|---|---|---|---|---|---|---|
| Characteristic | (N = 6) | | (N = 11) | | (N = 17) | |
| Mean (SEM) age, y | 32.3 | (2.9) | 35.8 | (3.6) | 34.6 | (2.5) |
| Mean (SEM) weight, kg | 72.9 | (7.0) | 74.7 | (5.0) | 74.0 | (4.0) |
| Type of hemophilia, n | | | | | | |
| A | 5 | | 10 | | 15 | |
| B | 1 | | 1 | | 2 | |
| Baseline disease severity, n (%) | 6 | (100) | 11 | (100) | 17 | (100) |
| Severe (<1% factor activity) | | | | | | |
| Mean (SEM) time since | 26.2 | (4.8) | 35.4 | (3.7) | 32.1 | (3.0) |
| diagnosis, y | | | | | | |
| Mean historical ABR (SEM) | 35.7 | (11.6) | 32.0 | (5.2) | 33.3 | (5.1) |

All participants in the study had severe hemophilia (<1% factor level). One participant in each dose group had hemophilia B and the remainder had hemophilia A. Age, weight, and number of bleeding episodes per year were broadly similar in both dose groups. A history of hepatitis C was reported in 11/17 patients in the study, however, no subjects were enrolled that were receiving treatment with ribavirin, interferon or other antiviral therapy. Additionally, subjects who had significant liver disease, including clinically significant cirrhosis per medical history or alanine/aspartate aminotransferase (ALT/AST) >3×ULN at screening, were excluded.

### Example 2: Safety of Fitusiran Treatment

An objective of the above-described study was to evaluate the safety of fitusiran in participants with hemophilia A or B with inhibitors. The safety analysis population included all the participants who had received at least one dose of fitusiran. Safety assessments included adverse event (AE) monitoring, clinical laboratory assessments (e.g., hematological, biochemical (including liver function tests), coagulation measurements [activated partial thromboplastin time (aPTT)/prothrombin time (PT), international normalized ratio, platelets, D-dimer, fibrinogen], and antidrug antibody formation [using a validated human enzyme-linked immunosorbent assay]), vital signs, and 12-lead electrocardiography. AEs and serious AEs (SAEs) were assessed throughout the study and coded according to the Medical Dictionary for Regulatory Activities (MedDRA®, version 16.0). AEs were graded based on their severity (mild, moderate, or severe) and the causal relationship to study drug or premedication recorded.

There were no fitusiran treatment discontinuations during the study, no thrombosis events, and no SAEs considered related to fitusiran. There were no instances of drug-induced anti-drug antibody formation. AEs were reported by the 17 (100%) participants, who had the maximum severity of mild or moderate in all cases. Table 3 below reports the AEs from all patients who received at least a single dose of fitusiran. Any drug-related AE reported in Table 3 refers to AE considered to be possibly or definitely related to the study drug. The most common drug-related AEs from Table 3 were reported during the study period in at least two participants.

TABLE 3

| | 50 mg | 80 mg | All treated | |
|---|---|---|---|---|
| n (%) | (N = 6) | (N = 11) | (N = 17) | |
| Any AE | 6 | 11 | 17 | (100) |
| Any drug-related AE | 5 | 7 | 12 | (71) |

| | | | |
|---|---|---|---|
| Serious AE | 0 | 1 | 1 |
| Serious drug-related AE | 0 | 0 | 0 |
| AE leading to discontinuation | 0 | 0 | 0 |

Most common drug-related AEs

| Injection site erythema | 3 | 5 | 8 | (47) |
|---|---|---|---|---|
| Alanine aminotransferase | 1 | 2 | 3 | (18) |
| increase | | | | |
| Aspartate aminotransferase | 1 | 2 | 3 | |
| increase | | | | |
| Fibrin D-dimer increase | 1 | 1 | 2 | (12) |
| Injection site pain | 2 | 0 | 2 | (12) |

The most common study drug-related AEs (occurring in ≥2 patients) were injection site erythema (n=8; 47%), ALT/AST increase, hepatic enzyme increase (alkaline phosphatase, bilirubin), or transaminases increase (n=5; 29%), fibrin D-dimer increase (n=2; 12%) and injection site pain (n=2; 12%). Injection site reactions were all mild and transient, and none required medical intervention. ALT elevations were generally mild and transient (four subjects with peak ALT≤3×ULN, one subject with ALT 5×ULN). There were no associated elevations in bilirubin greater than 2×ULN (reference range 3.4-20.5 μmon), and no severe drug-induced liver function impairment meeting Hy's Law criteria, which is considered predictive of whether a drug has the potential to cause severe drug-induced liver injury when given to a larger population (Katarey, et al., Clin Med (Loud.) (2016) 16(Suppl 6):s104-9).

Three participants had a history of hepatitis C virus (HCV) infection. The one case of ALT increase >3×ULN on Day 42 (5×ULN; 254.9 U/L) occurred in a participant (80 mg) with a history of HCV and was reported as moderate in intensity by the investigator; fitusiran dosing continued with subsequent ALT decrease to <3×ULN by Day 98 and resolved at the end of study. The remaining four cases of ALT elevations (<3×ULN) were resolved (n=1) or resolving (n=3) at the end of the study. Overall, the elevations in liver enzymes considered related to fitusiran were all asymptomatic, were assessed as mild or moderate by the investigator, and did not require fitusiran dosing suspensions or interruptions.

The majority of participants did not exhibit any shifts from baseline in coagulation parameters (e.g., PT, aPTT, D-dimer, and fibrinogen). There were no shifts in PT or aPTT that were reported as clinically significant by the investigators. Increased D-dimer were reported as AEs in three participants, two as possibly related and one as unlikely related. All elevations in D-dimer were considered mild in intensity by the investigator and were transient.

One participant (50 mg) experienced D-dimer increase (412 μg/L on Day 29 to 1349 μg/L on Day 42; reference range: 0 to 130 μg/L) that was considered possibly related to treatment, mild in intensity and was resolving at the end of study (526 μg/L). The participant had increased D-dimer (231 μg/L) prior to receiving fitusiran and the event was associated with moderate gastritis and mild ALT increase (<3×ULN).

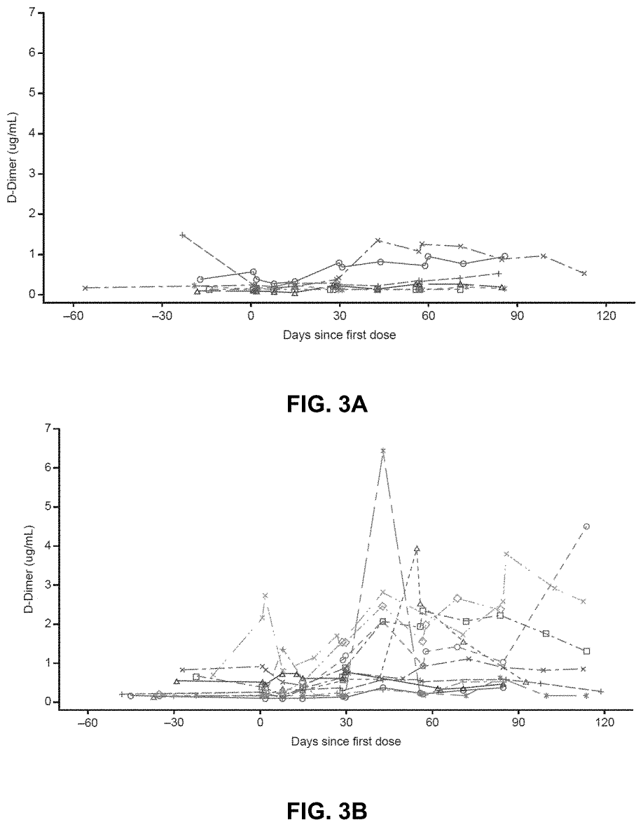

Another participant (80 mg) experienced D-dimer increase (2480 μg/L on Day 42; reference range: 0 to 590 μg/L) that was judged as possibly related to treatment and was mild in intensity. The participant had a history of HCV and the D-dimer increase was associated with mild elevation in ALT (<3×ULN). The patient was enrolled in the open label extension study, continued to receive a monthly fixed dose of fitusiran and did not have another episode of increased D-dimer, reported as an AE (FIGS. 3A and 3B).

A final participant (80 mg) with D-dimer increase (2090 μg/L on Day 42; reference range: 0 to 590 μg/L) was judged as unlikely related to fitusiran treatment and was mild in intensity. The participant had a history of HCV and elevation in D-dimer (690 μg/L) prior to receiving fitusiran. The event was associated with a nonserious AE of mild hepatic enzyme increase (considered related to treatment), though dosing was continued and the AE was resolved by the end of the study (450 μg/L). No association was observed between elevation in D-dimer and treatment of bleeds, liver enzyme abnormalities or fitusiran dose administered.

A total of four SAEs were reported by the participants in the study. One participant (18%) reported two SAEs of pneumonia and hamartoma, one had duodenal ulcer bleeding and one had muscle hemorrhage. None of the SAEs were considered related to the study drug, and all the SAEs were resolved.

### Example 3: Pharmacokinetics and Pharmacodynamics of Fitusiran Therapy

Another objective of the study described in Example 1 was to characterize the Pharmacokinetic (PK) of fitusiran and to assess the pharmacodynamic (PD) effects of fitusiran on AT activity and thrombin generation. The PK/PD population included all participants who had received at least one dose of fitusiran and had at least one plasma sample that could be evaluated.

For assess PK/PD analyses, plasma AT protein levels and thrombin generation were determined by an activity-based chromogenic assay (INNOVANCE® Antithrombin assay on an automated coagulation instrument; Siemens BCSxp; lower limit of quantitation (LLOQ) of 3.13 ng/mL) and a calibrated automated thrombogram assay (Thromboscope BV, Maastrict); an affinity fluorogenic substrate was used to measure the real-time analysis of tissue-factor triggered thrombin generation. The fluorescence was read with a Thermo Fluoroskan and reported as peak height, respectively. AT activity was measured in human plasma using a validated chromogenic assay for the quantification of functionally active AT calibrated against the AT activity of World Health Organization (WHO) reference plasma standard (LLOQ of 5% AT activity). Fitusiran PK parameters were calculated from plasma concentration-time data using non-compartmental analysis and Phoenix WinNonlin software.

1. Pharmacokinetics

Following the administration of single 50 mg and 80 mg doses of fitusiran, mean peak plasma levels of the drug (Cₘₐₓ) were 85.4 and 142.9 ng/mL on Day 0, and 96.5 and 157.1 ng/mL on Day 56 (after 3 monthly doses), respectively. In both dose groups, Cₘₐₓ was achieved approximately 4 hours post dose (Tₘₐₓ) for both assessment days (Day 0 and 56). Fitusiran levels decreased rapidly in plasma; the mean elimination half-life ranged from 3.4 to 5.2 hours, which was similar to results previously observed (Pasi, supra). Fitusiran PK parameters were similar in hemophilia patients with or without inhibitors.

Plasma PK parameters for fitusiran on Day 0 (after the first monthly SC dose) and on Day 56 (after 3 monthly SC doses) in hemophilia patients with inhibitors are provided in Table 4 below. AUC_{inf} in Table 4 refers to area under the curve extrapolated to infinity; AUCₗₐₛₜ refers to area under the curve to the last measurable concentration; CL/F refers to apparent clearance; Cₘₐₓ refers to maximum plasma concentration; CV refers to coefficient of variation; t_{1/2} refers to elimination half-life; tₘₐₓ refers to time to maximum plasma concentration; and Vz/F refers to apparent volume of distribution. Values for AUC_{inf} at D56 after repeat dosing were not reported.

TABLE 4

| | | | | | | | | |
|---|---|---|---|---|---|---|---|---|
| Fitusiran | | Cₘₐₓ | AUCₗₐₛₜ | AUC_{inf} | tₘₐₓ | t_{1/2} | CL/F | V_{z}/F |
| Dose | Statistic | (ng/mL) | (h · ng/mL | (h · ng/mL) * | (h) | (h) | (L/h) | (L) |

Day 0

| | | | | | | | | |
|---|---|---|---|---|---|---|---|---|
| 50 mg | N | 6 | 6 | 4 | 6 | 4 | 4 | 4 |
| | Geometric | 85.36 | 1053 | 1109 | 3.83 | 3.417 | 45.10 | 222.2 |
| | Mean | | | | | | | |
| | CV (%) | 42.1 | 39.8 | 45.4 | 79.4 | 37.6 | 56.1 | 95.6 |
| 80 mg | N | 11 | 11 | 9 | 11 | 9 | 9 | 9 |
| | Geometric | 142.9 | 3166 | 4011 | 3.70 | 5.049 | 19.96 | 145.5 |
| | Mean | | | | | | | |
| | CV (%) | 37.3 | 195.2 | 177.6 | 49.2 | 34.2 | 62.7 | 77.1 |

Day 56

| | | | | | | | | |
|---|---|---|---|---|---|---|---|---|
| 50 mg | N | 6 | 6 | — | 6 | 5 | 5 | 5 |
| | Geometric | 96.5 | 1118 | — | 4.06 | 4.343 | 41.36 | 259.2 |
| | Mean | | | | | | | |
| | CV (%) | 34.3 | 29.5 | — | 75.2 | 34.9 | 29.6 | 59.1 |
| 80 mg | N | 11 | 11 | | 11 | 10 | 10 | 10 |
| | Geometric | 157.1 | 1768 | — | 3.60 | 5.178 | 43.26 | 323.0 |
| | Mean | | | | | | | |
| | CV (%) | 32.9 | 23.1 | — | 67.7 | 25.2 | 22.1 | 44.6 |

2. Pharmacodynamics

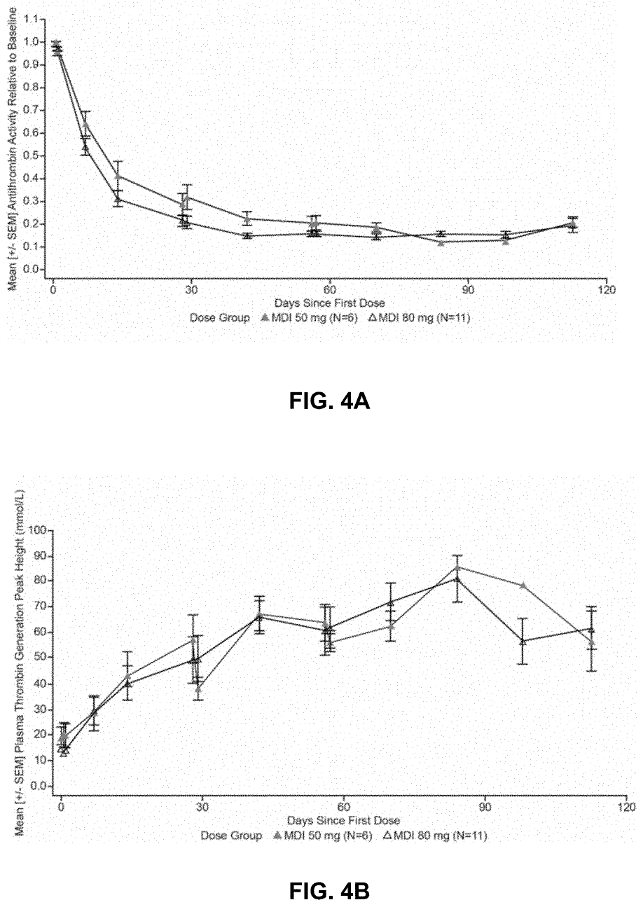

AT activity is measured in percent (normal range is approximately 80%-120% activity). Mean (standard error of the mean [SEM]) baseline AT levels were 109.5% (4.4) and 100.2% (4.8) of normal in the fitusiran 50 mg and 80 mg dose groups, respectively. There was consistent reduction in AT activity, evident initially at Day 7 and progressing to maximal effect after Day 28. The mean (SEM) maximum reduction in AT activity (as a percent change from baseline in AT levels) was similar between the dose groups, being 82.0% (2.2) in the 50 mg dose group and 87.4% (0.7) in the 80 mg dose group. The minimum residual post-dose AT level was 9.8%, observed in the 80 mg dose group. Mean (SEM) reductions from baseline in AT activity over time are shown in FIG. 4A. Reduced AT levels are associated with increased thrombin generation (FIG. 4B).

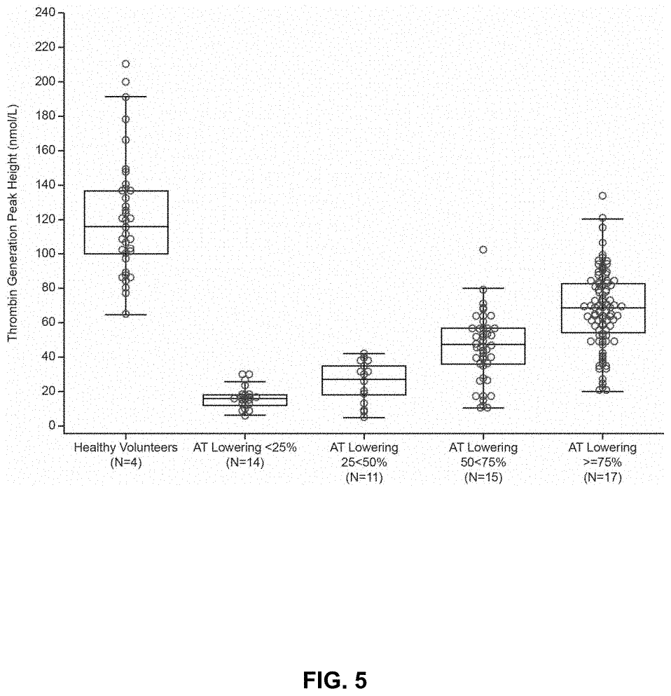

Thrombin peak height was associated with the degree of AT reduction in both dose groups and AT reduction by ≥75% from baseline resulted in a median peak thrombin height of 68.05 nM, which falls in the lower range previously observed in healthy volunteers (64-210 nM) (FIG. 5) (Pasi, supra).

### Example 4: Reduction of Annual Bleeding Rates by Fitusiran Therapy

One objective of the clinical study described in Example 1 was to evaluate the effect of fitusiran on annual bleeding rates (ABR). All patients were evaluated.

To assess the effect of fitusiran on ABR, detailed hemophilia history was collected and used to determine participants' historical ABR based on a 6-month period. During the study, all participants had a study-specific diary in which all episodes of bleeding, administration of BPA, and response to BPA treatment were recorded. Based on fitusiran's mechanism of action, the expected onset period to reach AT reduction of ≥75% is approximately 28 days. Onset period bleeding episodes were those that occurred from the first dose date and time of the study drug to Day 28 (inclusive). Observation period bleeding episodes were those that occurred from 4 weeks after the first dose (first dose date+29 days) until 8 weeks after the last dose of fitusiran (last dose date+56 days), or the last visit date in study, whichever was earlier. Causes and locations of bleeding episodes, and dose of factor and BPA treatments applied to each bleed, were captured.

Table 5 below sets forth the ABRs from participants in the study following administration of fitusiran once monthly, as compared to historical annualized bleed rates prior to fitusiran treatment. Historical ABR is the estimated number of bleeding events in the last 12 months. The Onset Period ABR was from Day 1 to Day 28 of the study, and the Observation Period ABR was from Day 29 to 8 weeks after last dose.

TABLE 5

| | | | | | Observation | |
|---|---|---|---|---|---|---|
| | Historical | | Onset Period | | Period | |
| | ABR | | ABR | | ABR | |
| | Mean | Median | Mean | Median | Mean | Median |
| Category | (SEM) | (range) | (SEM) | (range) | (SEM) | (range) |

By fitusiran dose

| | | | | | | |
|---|---|---|---|---|---|---|
| 50 mg | 33.67 | 33 | 17.39 | 13.04 | 15.12 | 6.52 |
| (N = 6) | (11.55) | (0.0, | (5.50) | (0.0, 39.1) | (7.98) | (0.0, |
| | | 80) | | | | 45.7) |
| 80 mg | 32.00 | 36.00 | 10.67 | 0.00 | 5.65 | 0.00 |
| (N = 11) | (5.22) | (8.0, | (5.51) | (52.2) | (4.07) | (0.00, |
| | | 54.0) | | | | 45.0) |
| All | 33.29 | 36.0 | 13.04 | 13.04 | 8.99 | 0.00 |
| treated | (5.09) | (0.0, | (4.03) | (0.0, 52.2) | (3.87) | (0.0, |
| (N = 17) | | 80.0) | | | | 45.7) |

The median ABR was lower than the historical median ABR for both dose groups during both the onset (Day 1-28) and observation (Day 29-8 weeks after last dose) periods (Table 5). The median observation period ABR for the 50 mg and 80 mg dose groups combined (n=17) was 0 (range 0-46), compared to a historical median ABR of 36 (range 0-80). The median (range) observation ABR was 7 (0-46) bleeds in the 50 mg dose group and 0 (0-45) bleeds in the 80 mg dose group, compared with pre-treatment rates of 33 (0-80) and 36 (8-54) bleeds, respectively (Table 5). Overall, 11/17 (65%) participants recorded 0 bleeding events while on fitusiran treatment and had an ABR of 0 during the observation period. Bleeds treated with BPA were successfully managed as assessed by the number of injections per bleed and participant ratings on the amount of BPA needed to control bleeds compared with bleeds prior to using fitusiran in the same location. When AT reduction was ≥75%, there were 6 bleeds that were treated with aPCC and 13 with rFVIIa. Overall, 83.3% (5/6) of the bleeds treated with aPCC were reported to use less aPCC than normal and 61.5% (8/13) of the bleeds treated with rFVIIa were reported to use less rFVIIa than normal to control the bleeding event. BPA dose ranged from 93-133 μg/kg for rFVIIa (median 108.6 μg/kg) and 14-75 U/kg for aPCC (median 28.6 U/kg).

### Example 5: Improvement of Patient-Reported Outcomes by Fitusiran Treatment

Another objective of the study was to evaluate patient-reported outcomes (PROs), including participant-reported hemophilia-related symptoms (painful swellings and presence of joint pain), physical health (pain with movement and difficulty walking far), and general quality of life. PROs are an important measurement of the efficacy of hemophilia therapy. In the study described in Example 1, PROs were collected using the Haem-A-QoL questionnaire and the EuroQol 5-Dimensions (EQ-5D) questionnaire. All patients were evaluated.

Haem-A-QoL domain scores and changes from baseline scores are reported in Table 6 below. Overall, all domain scores improved with treatment as demonstrated by mean changes from baseline scores (Table 6).

TABLE 6

| | 50 mg | | 80 mg | | Overall | |
|---|---|---|---|---|---|---|
| Haem-A-QoL domains | (N = 6) | | (N = 11) | | (N = 17) | |
| Total score | | | | | | |
| Mean (SEM) | −5.4 | (3.9) | −10.5 | (3.7) | −9.2 | (2.9) |
| Median | −2.4 | | −10.3 | | −8.6 | |
| N | 4 | | 11 | | 15 | |
| Sports and Leisure | | | | | | |
| Mean (SEM) | −5.4 | (5.3) | 1.9 | (7.0) | −0.2 | (5.5) |
| Median | −8.8 | | 5.0 | | 5 | |
| N | 3 | | 10 | | 13 | |
| Family planning | | | | | | |
| Mean (SEM) | 0.7 | (0.7) | −9.0 | (5.4) | −6.6 | (4.2) |
| Median | 0.0 | | −6.3 | | −3.1 | |
| N | 3 | | 9 | | 12 | |
| Feeling | | | | | | |
| Mean (SEM) | 6.3 | (7.7) | −20.5 | (8.0) | −13.3 | (6.9) |
| Median | 6.3 | | −18.8 | | −12.5 | |
| N | 4 | | 11 | | 15 | |
| Future | | | | | | |
| Mean (SEM) | −8.8 | (9.7) | −12.7 | (4.3) | −11.7 | (3.9) |
| Median | −5.0 | | −10.0 | | −10.0 | |
| N | 4 | | 11 | | 15 | |
| Dealing with Hemophilia | | | | | | |
| Mean (SEM) | 0.0 | (3.4) | −8.3 | (5.7) | −6.1 | (4.3) |
| Median | 0.0 | | −8.3 | | 0.0 | |
| N | 4 | | 11 | | 15 | |
| Partnership and sexuality | | | | | | |
| Mean (SEM) | 0.0 | (0.0) | −9.8 | (4.9) | −7.2 | (3.7) |
| Median | 0.0 | | −16.7 | | −8.3 | |
| N | 4 | | 11 | | 15 | |
| Physical health | | | | | | |
| Mean (SEM) | −5.0 | (3.5) | −15.0 | (5.1) | −12.3 | (3.9) |
| Median | −7.5 | | −15.0 | | −10.0 | |
| N | 4 | | 11 | | 15 | |
| Work and school | | | | | | |
| Mean (SEM) | −9.4 | (5.4) | −16.3 | (6.7) | −14.3 | (5.0) |
| Median | −6.3 | | −12.5 | | −12.5 | |
| N | 4 | | 10 | | 14 | |
| Treatment | | | | | | |
| Mean (SEM) | −10.2 | (6.0) | −12.5 | (5.3) | −11.9 | (4.1) |
| Median | −10.9 | | −15.6 | | −15.6 | |
| N | 4 | | 11 | | 15 | |
| View of self | | | | | | |
| Mean (SEM) | −11.3 | (4.3) | −6.8 | (4.2) | −8.0 | (3.3) |

| | | | |
|---|---|---|---|
| Median | −12.5 | 0.0 | 0.0 |
| N | 4 | 11 | 15 |

Notably, mean (SEM) changes from baseline to end of study (day 112) in Haem-A-QoL Total Score (−9.2 [2.9]) and Physical Health (−12.3 [3.9]) domain score (lower scores indicate better HRQoL) showed clinically meaningful improvements based on published thresholds (Wyrwich, supra). In addition, in the overall population, all domain scores improved with treatment as demonstrated by mean changes from baseline scores (Table 6). Further, the changes in PRO scores appear to be dose-dependent. In general, the effect size is greater in subjects on 80 mg vs. 50 mg dose.

The results indicate that fitusiran therapy has a beneficial impact on the patient experience and their quality of life, including joint health. It is also noteworthy that all other domains of the Haem-A-QoL demonstrated a numeric reduction (i.e., improvement) that appears to be dose-dependent, with the effect size stronger in the 80 mg group.

1. A method of improving joint function in a hemophilia patient, comprising subcutaneously administering fitusiran to a hemophilia patient in need thereof, optionally wherein the patient is a hemophilia A or B patient with or without inhibitors.

2. The method of claim 1, wherein the administering reduces difficulty in walking or increases mobility.

3. A method of improving a joint symptom in a hemophilia patient, comprising subcutaneously administering fitusiran to a hemophilia patient in need thereof, wherein the joint symptom is selected from joint swelling, painful movement, and joint pain, and optionally wherein the patient is a hemophilia A or B patient with or without inhibitors.

4. A method of improving patient-reported outcome (PRO) in a hemophilia patient, comprising subcutaneously administering fitusiran to a hemophilia patient in need thereof, optionally wherein the patient is a hemophilia A or B patient with or without inhibitors and optionally wherein the PRO is improved in one or more quality-of-life domains.

5. A method of improving quality of life (QoL) in a hemophilia patient, comprising subcutaneously administering fitusiran to a hemophilia patient in need thereof, wherein the QoL is improved in one or more QoL domains and optionally wherein the patient is a hemophilia A or B patient with or without inhibitors.

6. The method of claim 1, wherein fitusiran is administered at 40-90 mg per dose.

7. The method of claim 4, wherein the one or more QoL domains are domains in a QoL questionnaire, optionally wherein the QoL questionnaire is Haemophilia Quality of Life Questionnaire for Adults (Haem-A-QoL).

8. The method of claim 7, wherein the administering results in a clinically meaningful improvement indicated by a reduction of 7 or more units (optionally 8 or more, 9 or more, or 10 or more units) in one or more of Total Score, Sports and Leisure domain score, and Physical Health domain score of the Questionnaire.

9. The method of claim 1, wherein the patient is an adult or adolescent patient twelve years or older with hemophilia A or B with or without inhibitors.

10. The method of claim 1, wherein the patient has hemophilia A.

11. The method of claim 10, wherein the patient has been treated with factor VIII or a bypassing agent (BPA).

12. The method of claim 11, wherein the patient is with inhibitors, optionally wherein the level of inhibitors is more than 0.6 BU/mL as determined by Bethesda inhibitor assay.

13. The method of claim 10, wherein the patient is without inhibitors.

14. The method of claim 1, wherein the patient has hemophilia B.

15. The method of claim 14, wherein the patient has been treated with factor IX or a BPA.

16. The method of claim 15, wherein the patient is with inhibitors, optionally wherein the level of inhibitors is more than 0.6 BU/mL as determined by Bethesda inhibitor assay.

17. The method of claim 14, wherein the patient is without inhibitors.

18. The method of claim 11, wherein the BPA is activated prothrombin complex concentrates (aPCC) and/or recombinant activated Factor VII (rFVIIa).

19. The method of claim 1, comprising administering to the patient a plurality of doses of fitusiran at 50 mg per dose.

20. The method of claim 1, comprising administering to the patient a plurality of doses of fitusiran at 80 mg per dose.

21. The method of claim 1, wherein fitusiran is provided at a concentration of 50-200 mg/mL, optionally at a concentration of 100 mg/mL, in a phosphate-buffered saline (pH 7).

22. The method of claim 1, wherein fitusiran is administered to the patient once every four weeks or once a month.

23. An article of manufacture for use in the method of claim 1.

24-27. (canceled)


SEQUENCE LISTING (2 sequences)

SEQ ID NO: 1 - RNA, 21 nt, Artificial Sequence
  source: /note=“Description of Artificial Sequence Synthetic oligonucleotide”
gguuaacaccauuuacuucaa

SEQ ID NO: 2 - RNA, 23 nt, Artificial Sequence
  source: /note=“Description of Artificial Sequence Synthetic oligonucleotide”
uugaaguaaaugguguuaaccag